(19) United States
(12) Patent Application Publication
(10) Pub. No.: US 2013/0023865 A1
(43) Pub. Date: Jan. 24, 2013

(54) Imaging and Eccentric Atherosclerotic Material Laser Remodeling and/or Ablation Catheter

(71) Applicant: VESSIX VASCULAR, INC., Laguna Hills, CA (US)

(72) Inventors: Tom A. Steinke, San Diego, CA (US); Corbett W. Stone, San Diego, CA (US); Raphael M. Michel, San Diego, CA (US)

(73) Assignee: VESSIX VASCULAR, INC., Laguna Hills, CA (US)

(21) Appl. No.: 13/623,200

(22) Filed: Sep. 20, 2012

Related U.S. Application Data
(63) Continuation of application No. 11/122,263, filed on May 3, 2005, now abandoned.
(60) Provisional application No. 60/568,510, filed on May 5, 2004.

(51) Int. Cl. A61B 18/18; A61B 18/20

(52) U.S. Cl. 606/7

(57) ABSTRACT

Devices, systems, and methods for treating atherosclerotic lesions and other disease states, particularly for treatment of vulnerable plaques, can incorporate optical coherence tomography or other imaging techniques which allow a structure and location of an eccentric plaque to be characterized. Remodeling and/or ablative laser energy can then be selectively and automatically directed to the appropriate plaque structures, often without imposing mechanical trauma to the entire circumference of the lumen wall.

### CROSS-REFERENCES TO RELATED APPLICATIONS

This application is a continuation of U.S. patent application Ser. No. 11/122,263 filed May 3, 2005, which claims the benefit of priority from U.S. Provisional Application No. 60/568,510 filed May 5, 2004, and entitled “Imaging and Eccentric Atherosclerotic Material Laser Ablation Catheter,” both of which are incorporated herein by reference.

The subject matter of the present application is related to that of U.S. Provisional Application No. 60/502,515 filed on Sep. 12, 2003 for “Selectable Eccentric Ablation of Atherosclerotic Material” (Atty. Docket No. 21830-000100US); and to that of U.S. application Ser. No. 10/938,138 filed on Sep. 10, 2004 and entitled “Selectable Eccentric Remodeling and/or Ablation of Atherosclerotic Material,” the full disclosures of which are also incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

The present invention is generally related to medical devices, systems, and methods. In exemplary embodiments, the invention provides devices, systems, and methods which facilitate the controlled detection, characterization, and selective eccentric removal of atherosclerotic plaques in arteries via a laser catheter system. An exemplary apparatus combines optical coherence tomography plaque imaging with pulsed laser energy ablation.

### BACKGROUND OF THE INVENTION

Atherosclerosis is a major cause of cardiovascular disease. Atherosclerosis has traditionally been characterized by the progressive accumulation of atherosclerotic deposits (known as plaque) on the inner walls of the arteries. As a result, blood flow is restricted and there is an increased likelihood of clot formation that can partially or completely block or occlude an artery, often causing a heart attack.

Arteries narrowed by atherosclerosis are often now treated by medical procedures intended to increase blood flow. These procedures include highly invasive procedures such as coronary artery bypass surgery, and less invasive procedures such as balloon angioplasty, atherectomy, and laser angioplasty. Invasive bypass surgery can involve prolonged hospitalization and an extensive recuperation period, as well as the risk of major surgical complications. Less invasive options generally seek to avoid these disadvantages.

Balloon angioplasty is a less invasive and less costly alternative to bypass surgery. In this procedure, a balloon catheter can be inserted into a blood vessel through a small incision in the patient's arm or leg. The physician positions a balloon of the balloon catheter within the occluded area, often inflating and deflating the balloon several times. The inflation often tears the plaque and expands the artery beyond its point of elastic recoil. Although no plaque may be removed, the open lumen through which blood flows can be enlarged.

Atherectomy devices may provide symptomatic relief by both removal or ablation of the atherosclerotic plaque and improvement in vessel wall compliance through plaque fracture and excision. A relatively large minimal lumen diameter may be provided with atherectomy. A variety of atherectomy approaches have been pursued, including directional coronary atherectomy (DCA) and rotational atherectomy. Although they can remove some plaque from coronary arteries, existing atherectomy devices may be less effective in treating certain types of lesions. For example, rotational atherectomy often relies on differential plaque abrasion in which inelastic tissue (i.e., calcified plaque) is selectively abraded while elastic tissue (i.e., soft plaque) is deflected away from a rotation atherectomy burr. Not all atherosclerotic lesions are the same, however. For example, rotational atherectomy may be less effective in the treatment of softer atherosclerotic materials such as vulnerable plaques.

Vulnerable plaques and other atherosclerotic lesions do not necessarily conform to the occlusive accumulation model described above. In fact, many heart attacks may not be triggered by obstructions that narrow the arteries at all. Traditionally, coronary disease was thought by many to be akin to sludge building up in a pipe. Plaque can accumulate slowly, over decades, and once accumulated it was pretty much thought to be there for good. Every year, the narrowing was thought to grow more severe until one day no blood can get through and the patient has a heart attack. Bypass surgery or angioplasty—often, holding the vessel open with a stent—was intended to open up a narrowed artery before it closes completely. And so, it was assumed, heart attacks could be averted.

Many heart attacks may not be caused by an artery that is narrowed by plaque. Instead, heart attacks may often occur when an area of vulnerable plaque bursts, a clot forms over the area and blood flow is abruptly blocked. In a large percentage of cases, the plaque that erupts was not obstructing an artery sufficiently target the plaque for stenting or a bypass. This dangerous vulnerable plaque is often soft and fragile, may produce no symptoms, and would not necessarily be seen as an obstruction to blood flow. This may be why so many heart attacks are unexpected—a person will be out jogging one day, feeling fine, and may be struck with a heart attack the next. If a narrowed artery were the culprit, exercise might have caused severe chest pain. Vulnerable plaque may be identified using intravascular imaging, thermography (vulnerable plaque sometimes being referred to as “hot plaque”), and optical coherence tomography.

Proposals have been made to make use of laser energy in treatment of coronary artery disease. For example, rather than opening an artery relying entirely on mechanical balloon expansion, laser angioplasty may seek to thermally vaporize obstructions within the blood vessel, and more recently to selectively ablate plaques using wavelengths preferentially absorbed by atherosclerotic materials. To transmit sufficient laser energy, laser angioplasty catheters often include numerous thin optical fibers which may be bundled together or bound in a tubular matrix about a central catheter lumen. The laser energy emerging from a small number of fibers bundled together may produce small openings, and do not always remove an adequate quantity of matter from the lesion for use as a sole (or even primary) treatment. Laser angioplasty and similar devices may therefore be best suited for providing access through an occlusive plaque for subsequent conventional balloon angioplasty, rather than for treatment of vulnerable plaque.

Heart patients may have numerous vulnerable plaque lesions distributed in a variety of arteries. Drug therapies may seek to aggressively lower cholesterol levels, to get blood pressure under control, and to prevent blood clots throughout the patient's arteries. As such drugs end up distributed throughout the patient's tissues they often have deleterious side effects, and they may not produce the desired results in a timely manner for at least some patients. To effectively inhibit heart attacks, it may be advantageous to develop different treatment devices than those that are intended to target individual narrowed sections of one or more coronary arteries.

For the reasons given above, it would be advantageous to develop improved devices, systems, and methods for treatment of atherosclerotic materials.

### BRIEF SUMMARY OF THE INVENTION

The present invention generally provides improved devices, systems, and methods for treating atherosclerotic lesions and other disease states. While also being well-suited for treatment of occlusive diseases, the techniques of the present invention are particularly advantageous for treatment of patients who have (or are at risk of having) vulnerable plaques, regardless of whether those vulnerable plaques cause significant occlusion of an associated vessel lumen. Catheter systems of the present invention can incorporate optical coherence tomography or other imaging techniques which allow a structure and location of an eccentric plaque to be characterized. Ablative laser energy can then be selectively and automatically directed to the appropriate plaque structures, often without imposing mechanical trauma to the entire circumference of the lumen wall generally associated with balloon dilation, stenting, and known atherectomy methods.

In a first aspect, the invention provides a catheter system for remodeling and/or removal of atherosclerotic material from a blood vessel of a patient. The system comprises an elongate catheter having a proximal end and a distal end with an axis therebetween. The catheter has at least one window for transmission of laser energy near the distal end. At least one optical conduit extends between the proximal end of the catheter and the at least one window. An optical coherence tomographer or other analyzer is coupled to the at least one optical conduit. The tomographer may generates image signals using imaging light from within a plaque. The imaging light may be transmitted through the at least one window and proximally along the optical conduit. An ablation or remodeling laser is coupled to the tomographer or other analyzer, the laser transmitting plaque-remodeling and/or ablating laser energy to the at least one optical conduit in response to the signals.

The analyzer will often characterize the plaque and may also image the plaque, often using frequencies of light from the plaque to identify the tissue or atherosclerotic material type. Along with optical coherence tomography, spectroscopy (such as Raman spectroscopy) may be employed. The at least one window is often radially oriented for imaging and ablation of plaque eccentrically offset from the catheter relative to the axis. A first lens and a first mirror may be disposed along a first optical path between a distal end of the at least one conduit and the at least one window. A drive may be coupled to the proximal end of the catheter and a sleeve will often surround at least a portion of the optical conduit. The drive can effect scan the optical path relative to the sleeve, often by rotating the mirror about the axis. A first optical fiber bundle often directs the imaging light from the plaque to the tomographer and may also direct the ablation light toward the mirror.

In some embodiments, a second lens and a second mirror are disposed along a second optical path. A first optical fiber bundle can direct the imaging light from the plaque to the tomographer and a second optical fiber bundle can direct the remodeling and/or ablation light toward the mirror. The first and second optical paths adjacent the first and second mirrors can be circumferentially and/or axially offset. Optionally, at least a portion of one of the optical paths surrounds the other optical path. Alternative embodiments may make use of fluid core light guides in place of one or more optical fiber bundles.

In another aspect, the invention provides a catheter system for remodeling and/or removal of atherosclerotic material from a blood vessel of a patient. The system comprises an elongate catheter having a proximal end and a distal end with an axis therebetween. The catheter has at least one laterally oriented window disposed proximal of the distal end for radial transmission of optical energy. At least one optical conduit extends between the proximal end of the catheter and the at least one window. An analyzer is coupled to the at least one optical conduit, the analyzer generating signals using light from a plaque. The light is transmitted through the at least one window and proximally along the at least one optical conduit. A remodeling and/or ablation laser is coupled to the analyzer, the ablation laser transmitting plaque-remodeling and/or ablating laser energy to the at least one optical conduit in response to the signals so as to eccentrically ablate the plaque. The analyzer optionally comprises an imager such as an optical coherence tomographer, a tissue-characterizer such as an optical coherence reflectrometer, a Raman or other spectrometer, and/or the like.

In another aspect, the invention provides a method comprising advancing a catheter into a blood vessel and positioning the catheter so that an axis of the catheter extends along an atherosclerotic plaque. Imaging signals are generated from within the plaque using optical energy admitted radially into the catheter. In response to the imaging signals from within the plaque, plaque-ablating laser energy is transmitted eccentrically from the catheter.

In yet another aspect, the invention provides a method comprising advancing a catheter into a blood vessel and positioning the catheter so that an axis of the catheter extends along an atherosclerotic plaque. Signals are generated from the plaque using optical energy admitted radially into the catheter. In response to the signals from the plaque, plaque-remodeling laser energy is transmitted eccentrically from the catheter.

The signal generating step optionally comprises rotationally scanning an optical coherence tomographer, or the like, and may allow imaging of the plaque. The ablative laser energy can be selectively directed eccentrically in response to the imaging signals.

### BRIEF DESCRIPTION OF THE DRAWINGS

FIG. 1A illustrates diffuse atherosclerotic disease in which a substantial length of multiple blood vessels has limited effective diameters.

FIG. 1B illustrates vulnerable plaque within a blood vessel.

FIG. 1C illustrates the sharp bends or tortuosity of some blood vessels.

FIG. 1D illustrates atherosclerotic disease at a bifurcation.

FIG. 1E illustrates a lesion associated with atherosclerotic disease of the extremities.

FIG. 1F is an illustration of a stent fracture or corrosion.

FIG. 1G illustrates a dissection within a blood vessel.

FIG. 1H illustrates a circumferential measurement of an artery wall around a healthy artery.

FIG. 1I illustrates circumferential distribution of atheroma about a restenosed artery.

FIG. 2 schematically illustrates an atherosclerotic material imaging and remodeling and/or ablation catheter system according to an embodiment of the present invention.

FIG. 3 schematically illustrates laser light interacting with a tissue via absorption, surface reflection, internal scatter, and beam transmission

FIG. 4A graphically illustrates different laser absorption coefficients for a variety of tissues at varying wavelengths.

FIGS. 4B-4D graphically illustrate laser energy absorbance by tissues of the vascular system at varying wavelengths.

FIGS. 5A and 5B graphically illustrate depths and diameters, respectively, of ablations in atherosclerotic plaque using laser energy at varying powers.

FIG. 6 schematically illustrates an optical coherence tomographer imaging system for use in the catheter system of FIG. 2.

FIGS. 7A and 7B illustrate an intravascular optical coherence tomography image and an intravascular ultrasound image, respectively.

FIGS. 7C-7E illustrate Raman shift of plaque and images of associated tissues for a Raman spectroscopy system for use in the catheter system of FIG. 2.

FIG. 8 schematically illustrates a distal portion of a first embodiment of an imaging/ablation catheter for use in the catheter system of FIG. 2.

FIGS. 9A-9D and 10A-10D are cross-sectional images of the catheter of FIG. 8 being used within an artery for imaging, and for remodeling and/or ablation of atherosclerotic materials, respectively.

FIG. 11 schematically illustrates a second embodiment of an imaging/remodeling and/or ablation catheter for use in the catheter system of FIG. 2.

FIGS. 12A-12F are cross-sectional views showing the catheter of FIG. 11 being used within an artery to image and to remodel and/or ablate atherosclerotic materials.

FIG. 13 is a third embodiment of an imaging, and for remodeling and/or ablation catheter for use in the catheter system of FIG. 2.

FIGS. 14A-16F are cross-sectional view showing the use of the catheter of FIG. 13 (and related embodiments) being used for imaging, and for remodeling and/or ablation of atherosclerotic materials.

FIG. 17 schematically illustrates a fourth exemplary embodiment of an imaging/ablation catheter for use in the catheter system of FIG. 2.

FIGS. 18A-19F are cross-sectional view showing the use of the catheter of FIG. 17 (and related embodiments) for imaging, and for remodeling and/or ablation of atherosclerotic materials.

FIG. 20 schematically illustrates a fifth embodiment of an imaging, and for remodeling and/or ablation catheter for use in the catheter system of FIG. 2.

FIGS. 21A-21H are cross-sectional views showing the use of the catheter of FIG. 20 for imaging, and for remodeling and/or ablation of atherosclerotic materials.

FIG. 22 is a schematic view of a sixth embodiment of an imaging/ablation catheter for use in the catheter system of FIG. 2.

FIG. 22A is an end view of a concentric mirror for use in the catheter of FIG. 22.

FIGS. 23A-23H are cross-sectional views showing the use of the catheter of FIG. 22 for imaging, and for remodeling and/or ablation of atherosclerotic materials.

### DETAILED DESCRIPTION OF THE INVENTION

The present invention provides devices, systems, and methods to remodel and/or remove occlusive material from within body lumens, and particularly to safely remove or mitigate atherosclerotic material within a blood vessel while avoiding the release or embolization of clot-inducing and other deleterious substances. The techniques of the invention will often generate signals suitable for imaging, facilitating directing these treatments with reference to images displayed on a monitor. Nonetheless, while such signals might be used for (or be modified to be used for) generating an image, alternative embodiments might forego the monitor. Regardless, the signals may be used by an automated signal processing system to selectively transmit laser energy eccentrically from a catheter to an eccentric plaque along (for example) one side of a coronary artery, often by intermittently firing an ablative and/or remodeling laser at appropriate times during a rotational scan (such as when an optical path from the laser is aligned with the plaque).

While embodiments of the present invention may be used in combination with stenting and/or balloon dilation, the present invention may also be particularly well suited for mitigating vulnerable plaque and/or increasing the open diameter of blood vessels in which stenting and balloon angioplasty are not a viable option. The invention may provide particular advantages in treatment of vulnerable plaque or blood vessels in which vulnerable plaque is a concern, both by potentially identifying and avoiding inappropriate treatment of the vulnerable plaque, and by intentionally and selectively targeting vulnerable plaque for treatment using embodiments of the devices and methods described herein. In some embodiments, it may be possible to pierce a thin fibrous cap of a vulnerable plaque using ablation ablating and aspirate the cap and the lipid-rich pool of the vulnerable plaque, often within a controlled environmental zone or region within the blood vessel lumen. However, a vulnerable plaque is dangerous at least in part because the thin fibrous cap can break unexpectedly, allowing the lipid pool to propagate in the blood vessel and thereby creating thrombosis and clots. Thus, it may be advantageous in some embodiments to mildly heat a plaque which has been identified as a vulnerable plaque. Such mild heating may generate a reaction from the vessel that will lead to cap thickening, reducing the risk of the fibrous cap fracturing. Hence, such mild heating of a vulnerable plaque may transform the vulnerable plaque into a more mature, less vulnerable plaque. If a plaque is identified as an older, occlusive plaque, it may be desirable to heat the lipid pool so that it melts, migrates, and/or diffuses inside the artery wall, preferably reducing a thickness of the plaque.

At least some of the specific embodiments are described below with reference to devices and method suitable for ablation and/or removal of plaques. Other embodiments within the scope of the present invention may rely on alternative, and in some cases more gentle, treatment modalities. For example, rather than relying on an ablation laser, systems and methods similar to those described below may employ plaque remodeling lasers which do not effect ablation, and which instead pacify a vulnerable plaque. More generally, embodiments may employ light energy to remodel plaques, the remodeling often being done selectively so as to limit injury to adjacent tissues. As used herein, “remodeling” of plaques may comprise ablation, removal, shrinkage, melting, and/or the like, of the atherosclerotic plaques, and will usually modify the nature of the atherosclerotic plaque tissue (and consequently its size, shape, etc.) with the remodeling generally involving denaturing of the plaque.

There are several ways atherosclerotic tissue may be treated so as to open an at least partially obstructed vessel lumen. Examples of such treatments which are encompassed herein by the term “remodeling” include the use of mild laser energy (for example, at relatively low power) to heat up the atherosclerotic material until it melts. The liquefied material may then redistribute along the artery wall inside the vessel layers, often spreading out such that less material will be accumulated in one area. Such remodeled and redistributed plaque may be generally thinner so as to provide the vessel with an effectively larger lumen, improving blood flow.

Another remodeling modality that may be employed by other embodiments to treat atherosclerotic plaques may include the application of mild laser energy (for example, at relatively low power) to soften the atherosclerotic material. The blood pressure may then naturally push the softened plaque radially outward, resulting in a vessel with an effectively larger lumen, improving blood flow.

Still another remodeling modality that may be employed by other embodiments to treat atherosclerotic plaques may include the application of mild laser energy (for example, at relatively low power) to denature and shrink the atherosclerotic material. Shrinkage may be achieved by precise control of laser energy, and shrinkage of the atherosclerotic material may directly lead to a bigger vessel lumen and improved blood flow. As an example of remodeling by shrinking, when heated to around 85-90 degrees Celsius, a lipid pool of an atherosclerotic plaque may shrink and turn into fatty acids, which may be 90% smaller in volume than lipids. Those fatty acids may then be naturally evacuated through the capillaries of the artery wall. Preferably, the outer layer of the vessel (adventitia) will remain below 63 degrees Celsius during such heating to inhibit collagen shrinkage and vessel collapse, with the protection of these adjacent tissues often being achieved by precise control of the laser energy. The fibrous cap of a plaque (intimal layer) may thicken if heated to more than 50-60 degrees Celsius. Such an immune response to heating may lead to restenosis, so that such cap thickening and/or restenosis should also be limited by precise control of laser energy. Anti-restenotic drugs like Rapamycin and the like may also be employed.

Still other embodiments may remodel atherosclerotic plaques by directing sufficiently high laser energy (relatively high power) to ablate atherosclerotic material. If thrombotic ablation debris are generated, they may be constrained and/or evacuated by an aspiration lumen or other structure of the treatment catheters described herein, using a balloon, aspiration lumen or the like of a sheath surrounding the treatment catheters, by a separate catheter or filter structure, or the like. If the debris generated are non-thrombolitic, there may not be a need for catching and/or evacuation.

Still other embodiments may remodel plaques by altering the size or other properties of deleterious structures of the plaques. For example, some embodiments may provide advantages in treatment of vulnerable plaque or blood vessels in which vulnerable plaque is a concern, optionally by directing controlled laser energy toward such plaques so as to mildly heat the cap and/or lipid-rich pool of the vulnerable plaque to a temperature in a range from about 50 to about 60 degrees Celsius. Such heating may result in thickening of the cap and hence make the plaque less vulnerable to rupture, thereby effecting plaque stabilization.

Additional potential applications for embodiments of the present invention include treatment of diffuse disease, in which atherosclerosis is spread along a significant length of an artery rather than being localized in one area. Embodiments of the invention may also find advantageous use for treatment of tortuous, sharply-curved vessels, as no stent need be advanced into or expanded within the sharp bends of many blood vessel. Still further advantageous applications include treatment of the carotid artery along bifurcations, and in the peripheral extremities such as the legs, feet, and arms, where side branch blockage, crushing and/or stent fracture failure may be problematic.

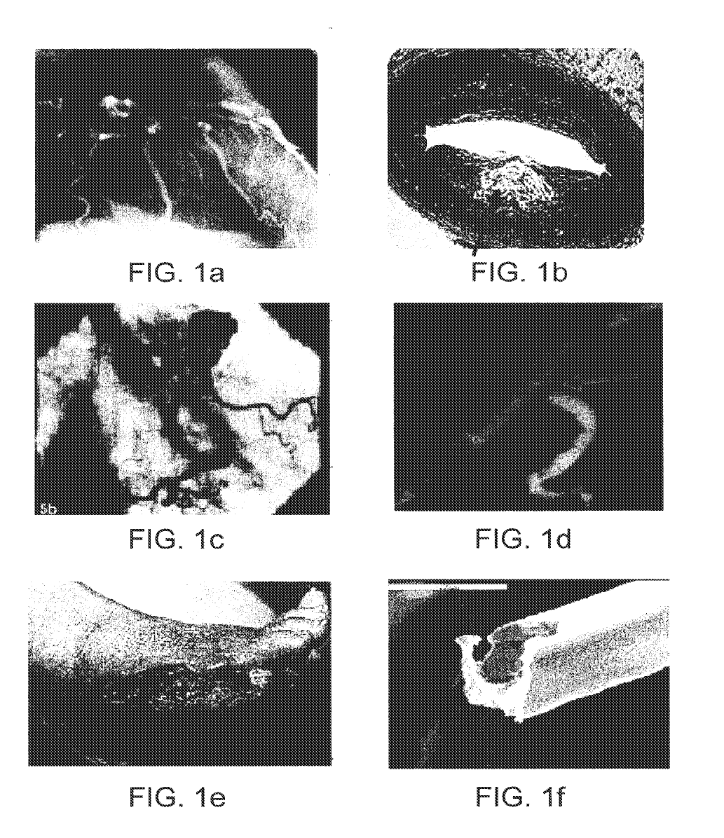

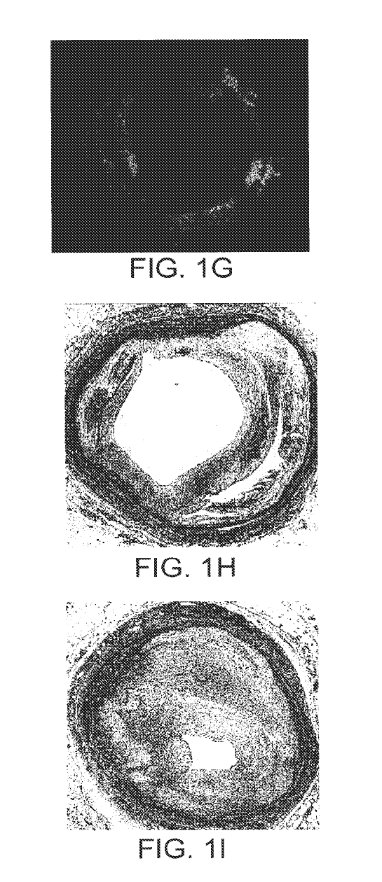

Diffuse disease and vulnerable plaque are illustrated in FIGS. 1A and 1B, respectively. FIG. 1C illustrates vascular tortuosity. FIG. 1D illustrates atherosclerotic material at a bifurcation, while FIG. 1E illustrates a lesion which can result from atherosclerotic disease of the extremities.

FIG. 1F illustrates a stent structural member fracture which may result from corrosion and/or fatigue. Stents may, for example, be designed for a ten-year implant life. As the population of stent recipients lives longer, it becomes increasingly likely that at least some of these stents will remain implanted for times longer than their designed life. As with any metal in a corrosive body environment, material degradation may occur. As the metal weakens from corrosion, the stent may fracture. As metal stents corrode, they may also generate foreign body reaction and byproducts which may irritate adjoining body tissue. Such scar tissue may, for example, result in eventual reclosure or restenosis of the artery.

Arterial dissection and restenosis may be understood with reference to FIGS. 1G through 1I. The artery comprises three layers: an intimal layer (including an endothelial layer), a medial layer, and an adventitial layer. During angioplasty, the inside layer may delaminate or detach partially from the wall so as to form a dissection as illustrated in FIG. 1G. Such dissections divert and may obstruct blood flow. As can be understood by comparing FIGS. 1H and 1I, angioplasty is a relatively aggressive procedure which may injure the tissue of the blood vessel. In response to this injury, in response to the presence of a stent, and/or in the continuing progression of the original atherosclerotic disease, the opened artery may restenose or subsequently decrease in diameter as illustrated in FIG. 1I. While drug eluting stents have been shown to reduce restenosis, the efficacy of these new structures several years after implantation has not been fully studied, and such drug eluting stents are not applicable in many blood vessels.

In general, embodiments of the present invention provide catheters which are relatively quick and easy to use by the physician. A catheter system of the present invention may allow occluded arteries to be opened to at least 85% of their nominal or native artery diameter. Rapid occlusive material removal may be effected using sufficient power to vaporize and/or photoablate tissues. The desired opening diameters may be achieved immediately after treatment by the catheter system in some embodiments. Alternatively, a milder ablation may be implemented, for example, providing no more than a 50% native diameter when treatment is complete, but may still provide as much as 80 or even 85% or more native vessel open diameters after a subsequent healing process is complete due to resorption of injured luminal tissues in a manner analogous to left ventricular ablation for arrhythmia and transurethral prostate (TURP) treatments. Such embodiments may heat at least some occlusive tissue to a temperature in a range from about 55.degree. C. to about 80.degree. C. Laser debulking, if complete (diameter stenosis >30%), may offer long-term restenosis results better than those of brachytherapy.

Advantageously, embodiments of the catheter systems and methods of the invention may be used without balloon angioplasty, thereby avoiding dissections and potentially limiting restenosis. Alternative embodiments may combine the structures and methods described herein with known angioplasty and stenting techniques.

The systems schematically illustrated in the attached drawings and described in this text may also be used in combination with a variety of known structures, with or without modifications to these known structures. For example, while generally described with reference to flexible catheter structures, alternative embodiments may make use of rigid catheter bodies or other rigid structures. Additionally, it may be advantageous to partially or fully isolate the blood vessel environment adjacent the distal portion of the laser ablation catheters described herein. Optionally, a lumen of an outer catheter having a toroidal balloon may receive any of the treatment/imaging catheters described herein so as to inhibit bloodflow. Similarly, the catheters described herein may include a central or offset lumen to accommodate a guidewire or the like, optionally a balloon guidewire so as to inhibit bloodflow distal to the treatment/imaging catheter. An outer catheter, the treatment/imaging catheters described herein, and/or a distal balloon-supporting guidewire may include at least one lumen coupled to an aspiration and/or irrigation source so as to provide a controlled ablation environment and inhibit the release of tissue fragments, atherosclerotic materials, ablation debris, and the like. At least some of the structures suitable for providing such an environment may be described in application 60/502,515, previously incorporated herein by reference.

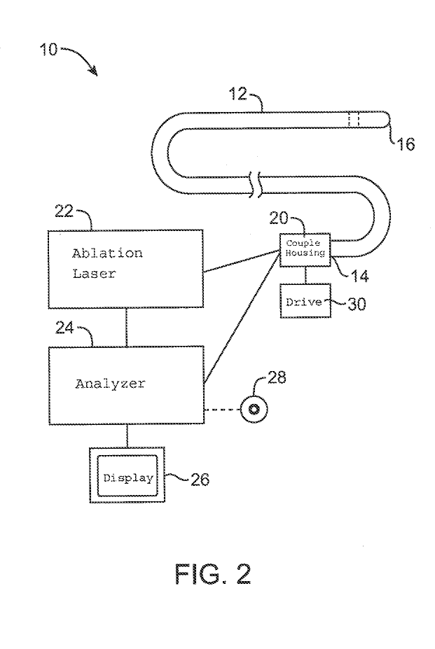

An exemplary imaging/ablation catheter system 10 is schematically illustrated in FIG. 2. An imaging/ablation catheter 12 has a proximal end 14 and a distal end 16, the catheter generally defining an axis 18. A housing 20 adjacent proximal end 14 couples the catheter to an ablation laser 22 and an analyzer 24, the analyzer often comprising an optical coherence tomography system. Optionally, a display 26 may show intravascular optical coherence tomography (or other) images, and may be used by a surgeon in an image-guided procedure. A drive 30 may effect scanning for at least one imaging component relative to a surrounding catheter sleeve, the scanning optionally comprising rotational scanning, helical scanning, axial scanning, and/or the like.

Additional system components, such as an input device for identifying tissues on the display for treatment and a processor for interpreting the imaging light signals from catheter 12 will often be incorporated into a laser or imaging system, or may be provided as stand-alone components. Analyzer 24 will optionally include hardware and/or software for controlling laser 22, drive 30, display 26, and/or the like. A wide variety of data processing and control architectures may be implemented, with housing 20, drive 30, laser 22, analyzer 24 and or display 26 optionally being integrated into one or more structures, separated into a number different housings, or the like. Machine readable code with programming instructions for implementing some or all of the method steps described herein may be embodied in a tangible media 28, which may comprise a magnetic recording media, optical recording media, a memory such as a random access memory, read-only memory, or non-volatile memory, or the like. Alternatively, such code may be transmitted over a communication link such as an Ethernet, internet, wireless network, or the like.

Catheter 12 will often be used to remove and/or remodel plaque using laser energy in any of a variety of wavelengths, often ranging from ultraviolet to infrared. This energy may be delivered from laser 22 to a lesion by a fiber optic light conduit of catheter 12. While continuous wave thermal lasers could be used to generate heat to vaporize plaque, alternative laser structures may have advantages for use in (for example) the coronary arteries. Hence, laser 22 may comprise an excimer laser. Excimer lasers use ultraviolet light to break the molecular bonds of atherosclerotic plaque, a process known as photoablation. Excimer lasers optionally use electrically excited xenon and chloride gases to generate an ultraviolet laser pulse with a wavelength of 308 nanometers. This wavelength of ultraviolet light can be absorbed by the proteins and lipids that comprise plaque, resulting in precise ablation of plaque and the restoration of blood flow while inhibiting thermal damage to surrounding tissue. The ablated plaque may be converted into carbon dioxide and other gases and minute particulate matter that can be easily eliminated.

Conventional light guides or conduits, similar to those used in laser angiography catheters, may be used to direct the laser energy from laser 22 to the targeted lesion using fiber optics. Individual optically conducting fibers may be made of fused silica or quartz, and can be fairly inflexible unless they are very thin. In order to bring a sufficient quantity of energy from the laser to the thrombus or plaque, catheter 12 may include a number of very thin fibers, each typically about 50 to 200 microns in diameter bundled together, the fibers optionally being bound in a matrix. Although individual fibers of such small dimensions are flexible enough to negotiate curves of fairly small radius, a bundle of such fibers is less flexible and more costly. Hence, catheter 12 may make use of an alternative to conventional optical fiber technology: the use of fluid core light guides to transmit light into the body, as discussed by Gregory et al. in the article “Liquid Core Light Guide for Laser Angioplasty”, IEEE Journal of Quantum Electronics, Vol. 26, No. 12, December 1990, and U.S. Pat. No. 5,304,171 to Gregory, both of which are incorporated herein by reference. Such fluid-core light guides may offer advantages in flexibility over fused silica fibers or bundles for accessing lesions through tortuous vessels.

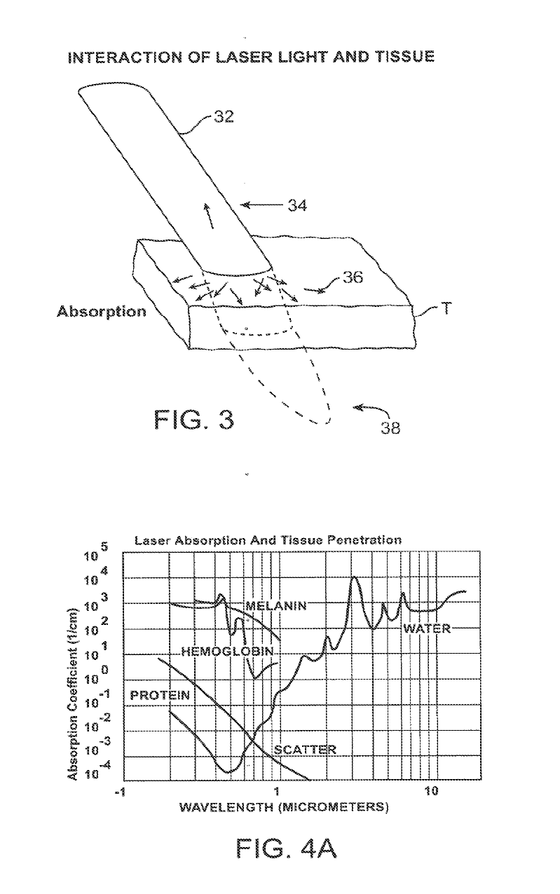

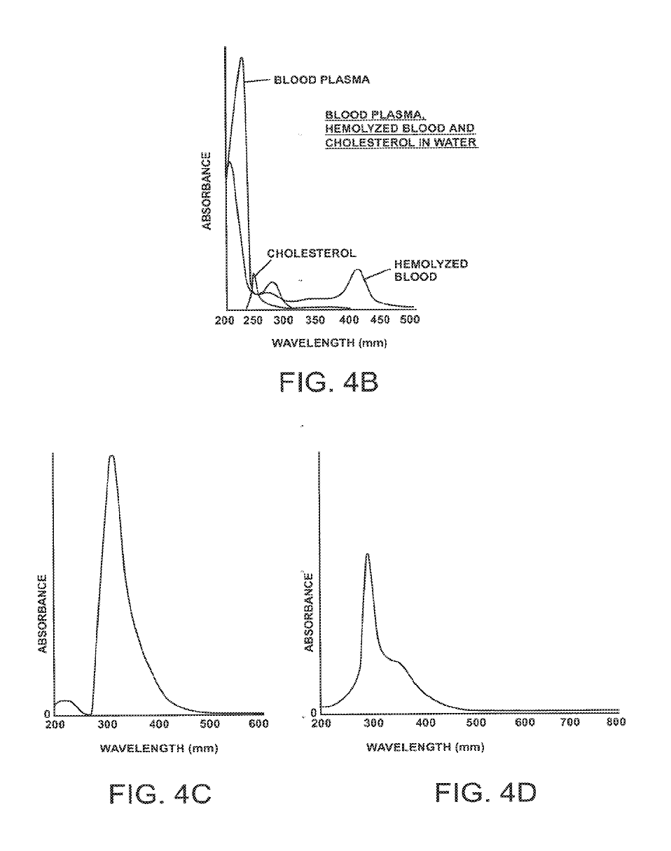

Referring now to FIG. 3, when a laser energy beam 32 strikes a surface of tissue T, four primary interactions can occur: surface reflection 34, scatter (including internal scatter 36), absorption, or transmission 38. The predominant interaction of many ablation lasers is absorption, which can cause tissue heating. Absorption by water can convert laser energy into heat. As can be understood with reference to FIG. 4, the degree of absorption can be tissue specific. Differing tissues have their own specific optical properties that determine the selectiveness and effectiveness of a particular laser.

Laser 22 may make use of a variety of structures to effect the desired tissue removal. Conventional lasers for bare fiber or “hot tip” laser angioplasty result in largely undirected thermal destruction. Excimer lasers often emit an ultraviolet beam that has sufficient energy to break intermolecular bonds (photoablation). Because little or no thermal damage occurs to adjacent tissue, this is often referred to as a “cool” laser beam. Excimer and other lasers are able to penetrate blood to a few millimeters in depth without loss of their ability to ablate tissue.

Still further ablative laser structures and wavelengths might be employed for laser 22, as can be understood with reference to FIGS. 4A to 4D. Peaks of the absorption spectrum in the ultraviolet region around 300 nm (usually 308 nm), and in infrared region such as at 2900 nm, suggest that lasers such as the XeCl excimer and the erbium YAG lasers, respectively, may be used as plaque ablators. 355 nm laser energy may also be employed for the removal of calcified plaque deposits, possibly inhibiting induced mutagenic changes in arterial tissue. The absorption of the ca. 244 to ca. 250 nanometers wavelength light by cholesterol (see FIG. 4C) is highly selective as compared to whole blood and healthy human blood vessel tissue, which exhibit little or no electromagnetic energy absorption peaks at or near these wavelengths. Atherosclerotic plaque has a similar absorbance peak between about 235 nm and 300 nm (see FIG. 4D).

Laser 22 may be either a continuous wave or pulsed laser. Continuous wave lasers often lead to deep thermal penetration with possible charring and shallow craters. In contrast, by providing sufficient time to permit thermal relaxation between pulses, a pulsed laser may reduce inadvertent heat conduction to surrounding tissues. Control of pulse duration and repetition rates can maximize the ablative properties of pulsed lasers as well as positively affect the particle size of ejected tissue.

While excimer lasers and other “cool” laser structures appear to provide significant advantages, alternative embodiments may intentionally cause ablation by raising the lesion temperature above the boiling point of water. Ablation can occur when a small volume of tissue is instantaneously heated above the boiling point of water. Water within the tissue is vaporized; remaining non-water components can be carried away in a plume of vapor and debris. The size of the particular debris may be affected by power and pulse characteristics of the laser. Hence, shallow penetrating, highly vaporizing laser can be used for system 10. Lasers such as the excimer holmium and erbium YAG are pulsed lasers may be available from many laser manufacturers, such as QUONTRONIX CORP. (Smithtown, N.Y.).

Charring may be avoided by thermally ablating only at power densities above a threshold. This surprising result, in which precise tissue ablation results from a higher rather than a lower power density setting, can help to minimizing thermal damage. Attempting to ablate tissue at lower power settings may risks greater thermal damage.

Surface thrombogenicity may be reduced after thermal plaque ablation. The loss of endothelium and exposure of subendothelial collagen may accelerate platelet deposition with risk of thrombus formation, and may initiate a proliferative response that could lead to restenosis. A pharmacologic therapy aimed at reducing platelet deposition, such as administering of coumadin, hirudin, argotropin, and hirulog may optionally be prescribed for the patient during the period of endothelial regeneration. Other pharmacological therapies may also be employed with the structures and methods described herein, including administering of streptokinase, urokinase, recombinant tissue plasminogen activators, heparin, or the like as described in U.S. Pat. No. 5,571,151.

Femtosecond lasers may also be adapted and/or used to ablate plaque and other atherosclerotic materials. Femtosecond lasers can use an infrared beam (for example, of 1053-nm wavelength) to cause photodisruption via laser-induced optical breakdown. The process of photodisruption may start when the fluence (energy/area) at the laser focus reaches a threshold that transforms matter in a normal state to a plasma (a high-density state of ions and free electrons). Temperature and pressure can increase rapidly in the opaque plasma because of the absorption of laser pulse energy, resulting in expansion. This in turn may create a shock wave and a cavitation bubble in which the tissue in the focal volume is destroyed. Femtoseconds lasers may operate at shorter pulse durations, and may therefore make use of less energy, produce smaller shock waves and cavitation bubbles than do the nanosecond Nd:YAG laser and the picosecond Nd:YLF laser.

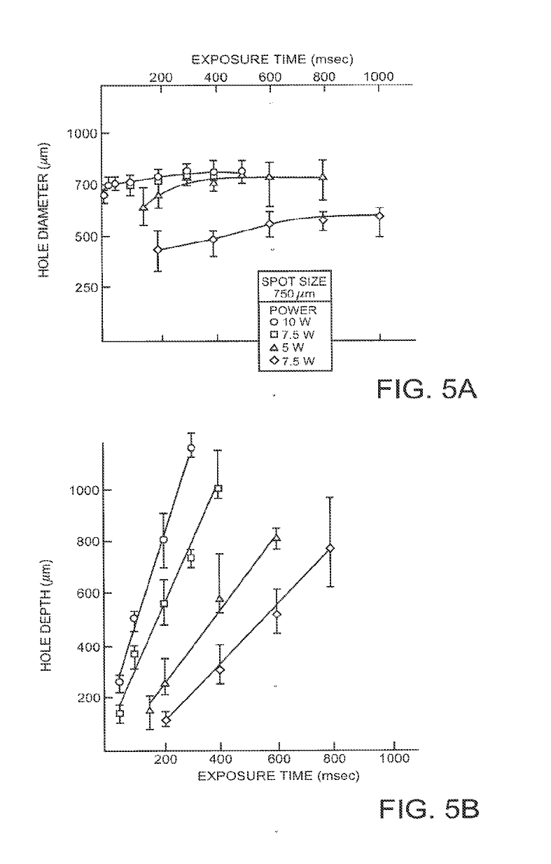

Firing of laser 22 can be automatically modulated in response to signals from analyzer 24 using signal processing software and/or hardware. Care should be taken to provide safe, reliable and precise guidance to energy from laser 22. First, determining precise depths of laser penetration will improve outcomes. System 10 may automatically control firing of laser 22 so as to remove atherosclerotic material while inhibiting ablation of healthy vessel tissues. FIGS. 5A and 5B are plots of depth and diameter, respectively, of holes formed by laser ablation in samples of atherosclerotic plaque with a 750 um spot size at various powers from about 2.5 W to about 10 W. Additional details on laser ablation depth may be found, for example, in U.S. Pat. No. 5,693,043, the full disclosure of which is incorporated herein by reference. Feedback of the effects of prior laser firings, as monitored by imaging system 24, can be used to enhance ablation depth and targeting control. The use of a proximal centering balloon or other centering structure may also enhance guidance accuracy, although such centering structures need not be included for use of some embodiments of catheter 12. Tracking of the laser catheter over a conventional guidewire may also enhance guidance of the laser delivery.

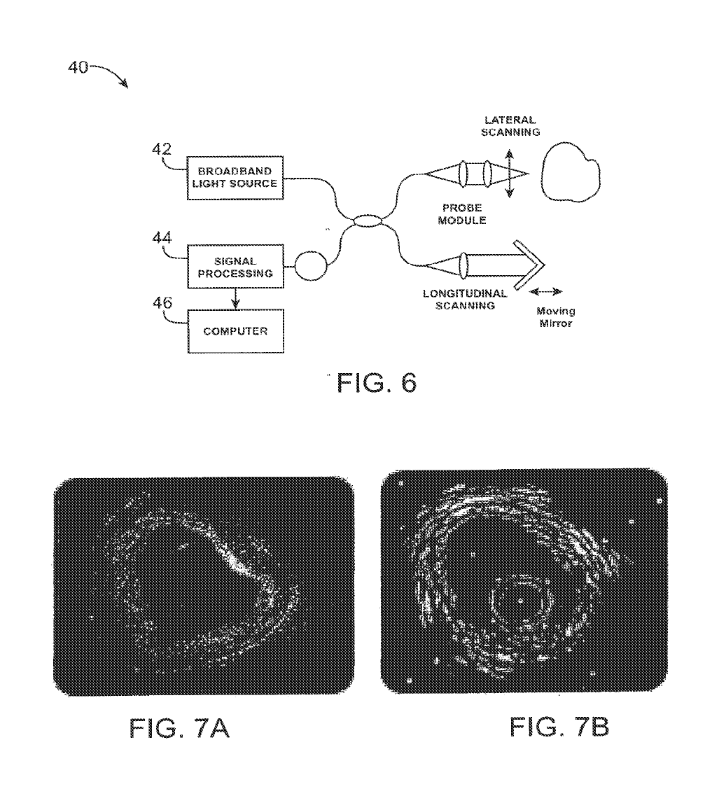

Referring now to FIGS. 2 and 6, in some embodiments, analyzer 24 of system 10 comprises an optical coherence tomography imaging system. Optical Coherence Tomography (OCT) utilizes advanced photonics and fiber optics to obtain images and tissue characterization within the human body Infrared light can optionally be delivered to the imaging site through a single optical fiber only 0.006″ diameter from broadband light source 42. Interferometric techniques can extract the reflected optical signals from the infrared light used in OCT in a signal processor 44. The output, measured by an interferometer, is computer processed to produce high-resolution, real time, cross sectional or 3-dimensional images of the tissue. This powerful technology provides in situ images of tissues at near histological resolution without the need for excision or processing of the specimen.

In addition to providing high-level resolutions for the evaluation of microanatomic structures OCT is able to provide information regarding tissue composition. Using spectroscopy, users and/or computer 46 can evaluate the spectral absorption characteristics of tissue while simultaneously determining the orderliness of the tissue through the use of polarization imaging. Targeted firing of the ablation laser may be in response to image signals indication location, shape, and/or composition of a plaque, often using automated image processing and spectral analysis programming, and optionally after verification and approval by the surgeon or other system operator. Alternatively, tissue characterization signals may be employed without imaging capabilities.

For many imaging systems (e.g., OCT imaging systems), light may be emitted from one or more single-mode optical fiber and focused on a sample using a lens. Retro-reflected light can then be coupled back through the lens into the fiber. In contrast to optical systems which rely on multimode optical fibers where the beam waist location and the classical image location are nearly coincident, in optical systems including single-mode optical fibers (which emit a nearly Gaussian beam), the waist location and the classical image location can be significantly different. This difference should be taken into account when designing lenses to be coupled with single mode optical fibers in order to attain the desired image location and depth of field.

In OCT and other imaging or light delivery/collection applications, the best optical performance is obtained when light impinges on a sample that is located within the depth of field of the lens. This improves efficiency for directing any light back-reflected from a sample through the single mode fiber. Light back-reflected farther and farther outside the working distance of the lens is received less and less efficiently by the single-mode optical fiber and hence is less detectable by the imaging system. Increasing the depth of field of the lens allows an optical conduit to image farther into a vessel or space into which the probe is inserted. The depth of field may be inversely related to the square of the beam spot size; thus, decreasing the beam spot size concurrently decreases the depth of field. With care, small optical systems may be designed to achieve both a large working distance and a large depth of field while still maintaining a small optical conduit diameter and small beam spot size.

In some embodiments, analyzer 24 may generate tissue characterization signals. Systems for generating such signals include reflectrometers and other devices which measure characteristics of light from an irradiated region so as to identify (and optionally locate or image) occlusive plaques, vulnerable plaques, and arterial walls. Near infrared light can be directed to the region, and may induce characterization light from the plaque via back-scatter, florescence, and/or the like, the characterization light being radially received by the catheter. Analyzer 24 may comprise a reflectrometer similar to the Optical Coherence Reflectrometer (OCR) developed by INTRALUMINAL THERAPEUTICS, INC. Optionally, system 10 will both image and characterize tissue surrounding the catheter by scanning laser and/or near infrared light circumferentially from the catheter as described herein.

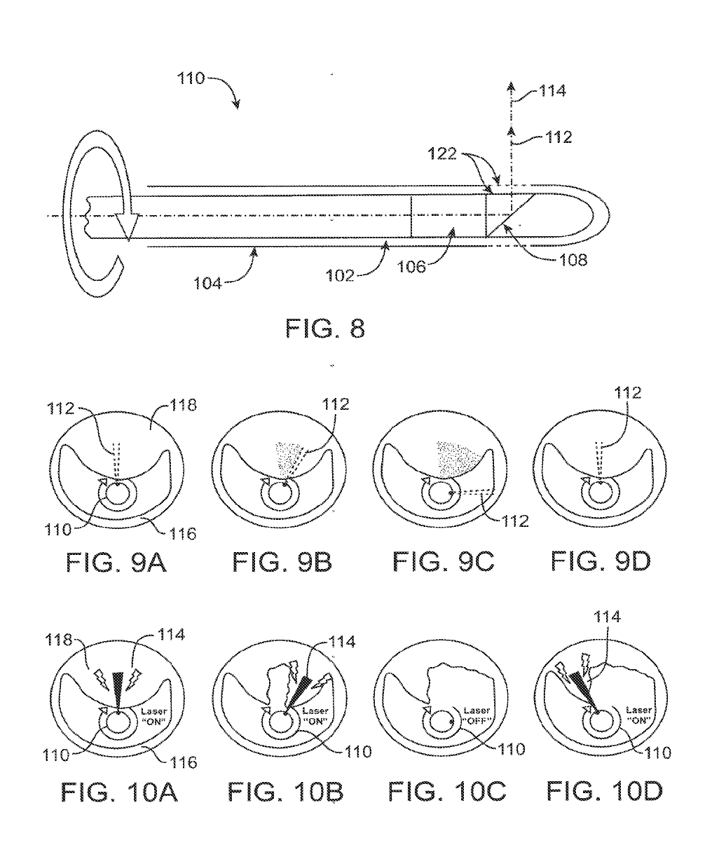

As can be understood with reference to FIGS. 6 and 8, in catheter 12 a single-mode fiber may be glued to a Graded Index (GRIN) lens using ultraviolet-cured optical adhesive (“UV glue”). The GRIN lens in turn can be UV-glued to a fold mirror, such as a prism, forming an optical chain comprising the single-mode optical fiber, the GRIN lens, and the fold mirror. The proximal end of the GRIN lens may be fixedly held within a rotatable torque cable. The entire assembly (i.e., optical chain and torque cable) may be contained within a catheter sleeve or sheathing. The sheathing is typically transparent to the wavelength of light contained with the single-mode fiber or includes one or more transparent window near the fold mirror. An ultra-small optical imaging probe that can perform circumferential imaging of a sample is described in more detail in U.S. Pat. No. 6,552,796 (incorporated herein by reference), which also describes methods of manufacturing the micro-optical elements (e.g., microlenses and beam directors) that form the distal imaging optics of such a probe. More specifically, miniature lenses which include the following optical properties were described in that reference, and may be employed between the optical conduit and the fold mirror of catheter 12:

A lens 2 diameter of less than about 300 μm (preferably less than about 150 μm);

A working distance >1 mm;

A depth of field >1 mm;

A spot size of <100 μm;

Ability to work within a medium with an index of refraction >1 (e.g., within a saline or blood-filled environment) without destroying the image quality;

Ability to rotate or perform circumferential scanning within a 400 μm diameter housing;

Ability to achieve >20% coupling efficiency from a fold mirror 3 located at the beam waist location of the lens 2; (Coupling efficiency is defined here as the amount of light energy recoupled or redirected by the lens 2 system back into the fiber 1.)

Minimal Back-Reflections.

Optical Coherence Tomography has several advantages, including a high resolution, ability to characterize tissues, small size, at or near real time imaging, and ability to provide Doppler imaging flow measurements. Current OCT systems may have resolutions at 4-20 μm compared to 110 μm for high frequency ultrasound. Using information from the returning photon signals, OCT can provide both spectroscopic and polarization imaging to better evaluate the composition of tissues and lesions. While OCT has the potential to be used for a variety of medical applications, cancer and heart disease represent two promising application areas. OCT has the potential to characterize plaques and help differentiate unstable vulnerable plaques from standard occlusive plaques.

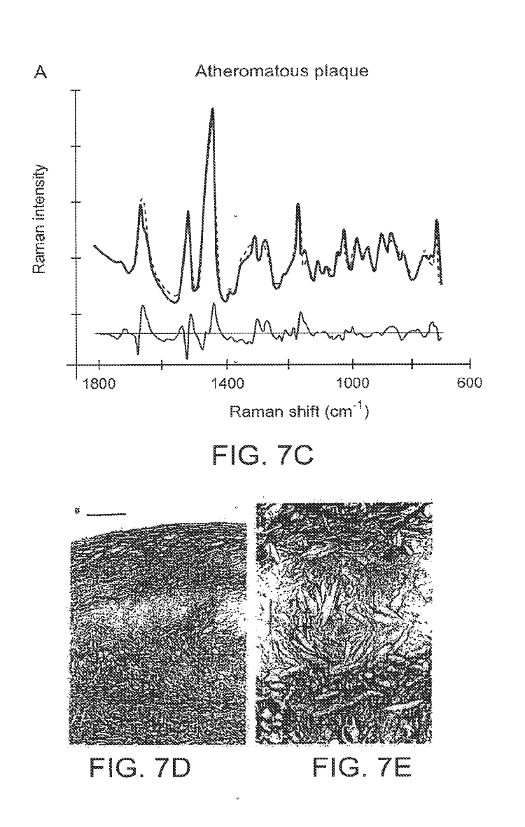

Many cancers may originate in the epithelium, the thin (20-200 micron) cellular layer covering the inner and outer surfaces of the body. Excisional biopsy, removing tissue from the body and examining it under a microscope can be effective for cancer diagnosis. However, OCT has the potential to greatly improve conventional biopsy by more precisely identifying the areas to be excised based on images of the epithelial layers, reducing the number of biopsies and making earlier and more accurate diagnosis possible. OCT systems, technology, and components may be commercially available from Humphrey Instruments (a subsidiary of Carl Zeiss, Inc.); the Pentax® Medical Instrument Division of Asahi Optical Company, Ltd.; LightLab Imaging; and Lantis Laser, Inc. for macular degeneration, endoscopic Optical Coherence Tomography for intravascular, gastrointestinal and pulmonary applications, dentistry and the like. FIGS. 7A and 7B provide a comparison between intravascular OCT imaging (FIG. 7A) and intravascular ultrasound imaging (FIG. 7B). Exemplary apparatus and methods for selective data collection and signal to noise ratio enhancement using optical coherence tomography are described in U.S. Pat. No. 6,552,796, the full disclosure of which is incorporated herein by reference.

Referring now to FIGS. 7C-7E, still further alternative analyzer structures may be employed to characterize plaque and other tissues from light frequencies and the like therefrom. For example, intravascular characterization and or imaging of atherosclerotic tissues may be achieved using Raman spectroscopy. FIG. 7C graphically illustrates Raman shift spectra for a plaque, while FIGS. 7D and 7E show the corresponding constituents of the atherosclerotic plaque. Structures and method for employing Raman spectroscopy to characterize tissues may be more fully described in an article entitled “Histopathology of Human Coronary Atherosclerosis by Quantifying Its Chemical Composition With Raman Spectroscopy” by Tjeerd J. Romer, MD et al. in Circulation 1998; 97:878-885. As described above, the signals generated by these and other analyzers may be used to selectively treat plaques while inhibiting injury to adjacent tissues.

While generally described herein with reference to the vasculature, embodiments of the catheter devices, systems, and methods described herein may also find applications in the lumens of other vessels of the human anatomy. The anatomical structure into which the catheter is placed may be for example, the esophagus, the oral cavity, the nasopharyngeal cavity, the auditory tube and tympanic cavity, the sinus of the brain, the larynx, the trachea, the bronchus, the stomach, the duodenum, the ileum, the colon, the rectum, the bladder, the ureter, the ejaculatory duct, the vas deferens, the urethra, the uterine cavity, the vaginal canal, and the cervical canal, as well as the arterial system, the venous system, and/or the heart.

Embodiments of the structures and methods described herein may be suitable for physical targeting and/or frequency targeting of selected tissues. Physical targeting of eccentric disease, for example, can be accomplished by positioning a window or other optically transmitting element relative to the target tissue, often by moving at least a portion of a catheter longitudinally within a lumen vessel until an optical path of treatment energy is oriented toward or in the vicinity of the targeted tissue. An additional method to physically target eccentric disease is to apply intermittent energy while rotating an optical path-defining component of the catheter, such as a fiberoptic conduit, mirror, and/or working window so as to selectively direct energy toward the targeted tissue, and so as to inhibit injury to healthy tissue.

To enhance the remodeling efficacy and/or limit collateral damage, embodiments of the devices, system, and methods described herein may tune the laser energy to the atherosclerotic materials to be treated. Characteristics of the laser energy, including the frequency, power, energy, delivery time, delivery location, and/or patterns or combinations thereof may be predetermined before diagnosis or treatment of a specific patient, the energy characteristics being transmitted without feedback, such as by employing open-loop dosimetry techniques. Such predetermined characteristic tuning may be based on prior laser irradiation of atherosclerotic materials, prior clinical trials, and/or other development work. Some embodiments may tune the laser energy directed to a particular patient based on in situ feedback, and many embodiments may employ some predetermined characteristics with others being feedback-controlled.

Embodiments may employ frequency targeting, often by taking advantage of different tissue types having different wavelength absorption characteristics. These differences can help the target tissue to absorb energy of certain frequencies or frequency ranges more readily than others. By applying energy at a frequency or within a range of frequencies that the diseased tissue can more absorb, and often at or within which adjacent tissues are less absorbent, energy penetrates to the target tissue and/or selectively heats the target tissue more readily.

Frequency targeting can help to deliver a greater portion of the transmitted energy to diseased tissue by identifying the frequency or range of frequencies at which the optical absorbance of the diseased tissue is at or near a peak, at or near a local peak, a practical maximum given the ease of generating laser energies, and/or equal to that of the adjacent healthy tissue. In some cases, energy absorbance of the plaque or the like may be less than that of adjacent healthy tissues. Energy delivered at the specified frequency or range of frequencies will often cause more heat to be directed to the diseased tissue than energy delivered outside of those specific frequencies.

Optical measurement (optical coherent tomography, Raman spectroscopy, and the like) can also be used to determine a state of a tissue. The selective optical absorption and/or reflectivity can characterize the molecular state of a tissue, including states which can be affected/changed by temperature. For example, lipids may start denaturing at 85 C, often turning into a new state, fatty acids. This new state can be as much as 90% more compact in volume than the original lipids. As the temperatures of such state changes for tissue are often known, and as the optical characteristics of the different states of the tissue can be identified, then by measuring the tissue optical characteristics, a state change and/or a temperature (such as a temperature estimate, profile, or the like) may be generated from the optical signals.

In some embodiments, specific frequencies may be employed to verify tissue type and/or condition of tissue based on optical measurement. The localization, identification, diagnosis, discovery, and/or characterization of diseased tissue can be provided using OCT imaging or other methods. Measurement of tissue optical characteristics radially may also allow for verification of the existence and classification of diseased tissue types.

Still further embodiments may be beneficial, including those employing multiple frequency therapies. The tissue remodeling therapies described herein can comprise the application of optical energy at a single frequency or at multiple frequencies. Depending on the composition of the target tissue and surrounding tissue, the optimum treatment may consist of a single frequency to target a single tissue type, multiple frequencies to target multiple tissue types, or multiple frequencies applied to a single tissue type. Multiple frequencies can be applied in any sequence, and can be applied as discrete frequencies or can be applied as a frequency sweep across a range in a linear, logarithmic, or other manner.

A variety of energy control techniques may be employed to help set up a correct initial dosage. The shape and type of diseased tissue to be treated is generally diagnosed and characterized by ultrasonic, optical, or other types of intraluminal sensing devices. Optical measurements can be used to understand the optical absorbance and/or other optical characteristics of atherosclerotic tissue of varying geometries and types. Using the optical characteristic data, the initial therapy dosage setting might be optimized.

Controlling the dosage may also be facilitated by signals from the analyzer. The optical absorbance characteristics of tissues may vary with temperature variations and/or the molecular state of a tissue. Dynamic measurement of optical absorbance of the tissue during application of energy can be used in a control feedback system to monitor and/or control the temperature changes of tissue. Related techniques may be implemented to help determine a desired or proper dosage during therapy. The pattern of energy delivery can be a single pulse or multiple pulses of varying duration, with the energy delivery optionally being separated by periods of varying duration. The measurement of optical absorbance of the tissue during energy delivery and between energy pulses may be used to determine the optimum durations of energy delivery and intervening or resting periods.

Optionally, pre-treatment bursts or pulses of laser energy can be applied to condition the target tissue for a desired treatment. Such pre-conditioning may be utilized, for example, to activate Heat-Shock Proteins (HSPs) in healthy tissue prior to treatment to help inhibit injury to the healthy tissue. Post-treatment bursts or pulses of laser energy may be applied, for example, to control the cool-down time of the tissue. Interim treatment bursts or pulses of laser energy may be applied, for example, to control the temperature profile of the target and/or surrounding tissue between multiple therapy bursts or pulses. Energy may, in differing embodiments, be delivered in a wide variety of combinations of amplitude and frequency.

Analyzer 24 may employ still further techniques to provide tissue temperature measurements. For example, optical absorbance measurements taken prior to therapy may be used as (and/or to calculate) a normalized value. Subsequent measurements may be used (optionally in further calculations) to determine the change in temperature from the initial values. Optionally, dynamic monitoring of the optical absorbance of target and surrounding tissue during therapy may be utilized to calculate the change in temperature of tissue. These or other temperature changes during therapy can be utilized to determine the effectiveness of energy delivery settings, and/or to determine the condition of the tissue being treated. Temperature measurements may be performed by intraluminal ultrasound, electrical impedance, or other mechanisms, and/or any temperature measurements may be verified by (or used to verify) data derived from optical absorbance measurements. Where it is desired to make use of electrical measurements, blood may optionally be used as a contact interface. Blood is a conductive ionic fluid that may be used as an interface between electrodes and tissue to ensure a good electrode-tissue contact and low contact impedance.

Closed loop control of different types may be included in some embodiments. Optical absorbance or other measurements, optionally over a plurality of frequency ranges and/or across multiple electrodes can be utilized to monitor and to verify physical changes such as tissue shrinkage or denaturing of tissue in the therapeutic energy application area. This data can be utilized to verify physical changes observed by other intraluminal observation techniques such as OCT implemented in analyzer 24. Data from optical absorbance and/or other measurements may be combined with inputs from intraluminal measurement devices such as OCT, and may be used to determine location and characteristics of treatment from a predetermined set of rules. Such a feedback control system may provide an automatic mode to diagnose and treat diseased intraluminal tissue. Data about the condition of the tissue, including temperature change, tissue optical absorbance, intraluminal geometry, and/or tissue type signal generated by analyzer 24 using OCT or other techniques can be utilized as inputs to a closed loop control system.

Referring now to FIGS. 2 and 8, catheter 12 generally uses one or more bundles of one or more rotatable optical conduits (sometimes referred to as “optical probes” herein) to direct light energy towards an artery wall at a given angle. The optical conduits may comprise one or more single-mode optical fiber, and may be housed inside a sleeve catheter or guidewire. The optical conduits may, at least in part, define optical paths, and each optical path may also be defined by a lens 106, and a fold mirror 108. The optical conduits are used to convey light energy for imaging and ablating. The corresponding light energies can be referred to as “imaging light” and “remodeling and/or ablating light.” While generally illustrated being directed from catheter 12, the imaging light may also be received by and transmitted proximally along the body of catheter 12 using the same optical conduit that transmits the imaging light and/or the ablating light distally, or using a separate optical conduit.

As can be understood with reference to FIG. 2, imaging system 24 provides an intra-vascular high-resolution image of the artery wall and allows for detection and identification of atherosclerotic plaques. When a plaque is identified and localized by imaging, an ablating light is pulsed through a rotatable optical conduit in such a way that it hits the plaque specifically, but does not damage the healthy area of the artery. The imaging can be done by Optical Coherence Tomography (OCT). The catheter system may or may not comprise a centering structure to maintain catheter 12 centered within an open lumen of the vessel adjacent the treatment delivery and/or imaging window(s).

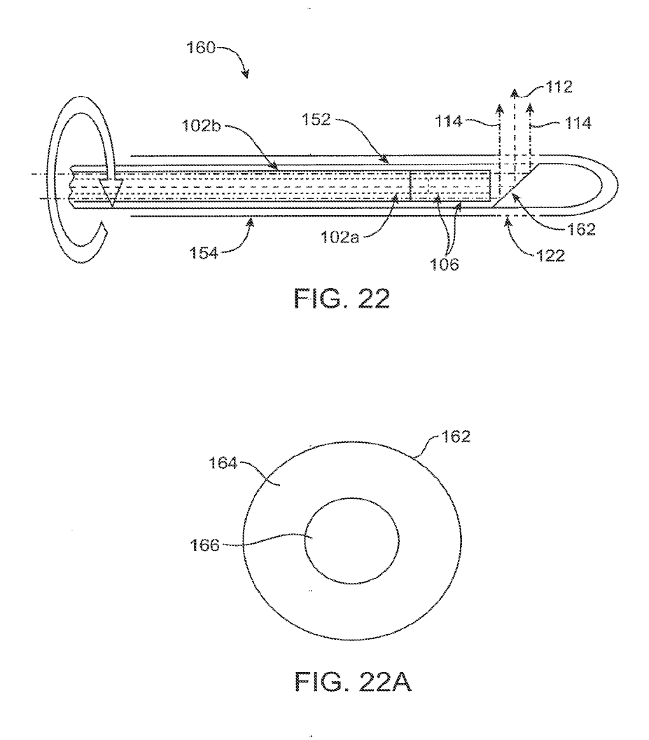

In some embodiments, only one optical conduit, or one bundle of the same optical conduits, may carry both the imaging light and the ablating light. Nevertheless, the wavelengths of the imaging light and the ablating light can be different, for instance by using a splitter (see attached U.S. Pat. Nos. 5,304,173 and 6,120,516 for a more detailed description of a splitter). The power of the light energy for imaging and ablation can also be different, for instance by using two different sources, or by using a single source that can be variable (see U.S. Pat. No. 5,304,173). The size and shape of the areas of imaging and ablation can be different, for instance by using different lenses and/or mirrors, explained with reference to preferred embodiment as explained in preferred embodiment 160 of FIG. 22. The shape of the light beams to perform imaging and ablation can be different, for instance by using different lenses and/or mirrors. For these reasons, the optical conduits that carry the imaging light and the ablating light can be different to provide the desired performance. For instance, the fiber optics can have a different mode, the characteristics of the lenses can be different, the fold mirrors can have different shapes, reflectivity, etc., as explained with reference to preferred embodiment 160. Hence, in some preferred embodiments, the same optical conduit can be used to carry both imaging and ablating light. In other preferred embodiments, two different optical conduits can be used to carry the imaging light and the ablating light. The imaging and ablation may be sequential or simultaneous.

A preferred embodiment 110 can be understood with reference to FIG. 8. In this preferred embodiment, the same optical conduit or bundle of optical conduits 102 is used to convey the light energy for imaging, say imaging light 112, and the light energy for ablating atherosclerotic plaques, say remodeling and/or ablating light 114. The optical conduits are housed inside a sleeve catheter or guidewire 104.

As can be understood with reference to FIGS. 8, 9A-9D, and 10A-10D, the optical conduits rotate continuously inside the sleeve catheter. The imaging light runs through the optical conduits and radially through transparent cylindrical windows 122 to provide an intra-vascular image of artery 116, for instance by OCT. The image is processed by a computer that identifies and localizes atherosclerotic plaques 118. Based on the information from the imaging, the computer then determines when to fire the ablating light 114 such that the light ablates specifically the plaque and does not damage the healthy area of the artery. This may be done by pulsing ablating light 114 on the plaque when the rotatable optical conduits face the plaque. The ablating light runs through the same optical conduits as the imaging light. The imaging and ablating lights are used sequentially.

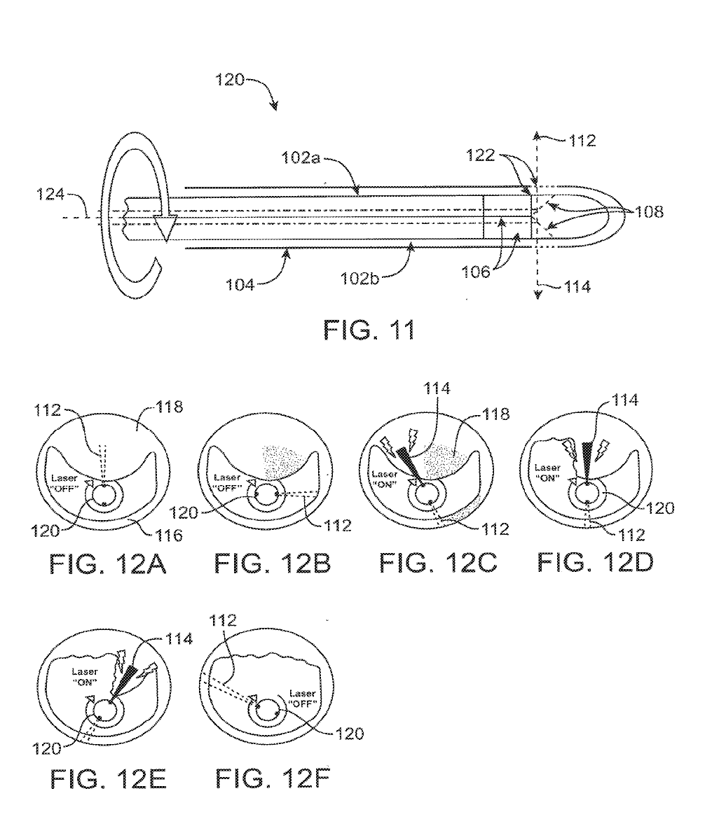

A preferred embodiment 120 can be understood with reference to FIG. 11. Two different optical conduits or bundles of optical conduits, 102a and 102b, are used to convey the light energy for imaging, say imaging light 112, and the light energy for ablating and/or remodeling atherosclerotic plaques, say ablating light 114. The optical conduits are housed inside a sleeve catheter 104 or guidewire. As shown in FIG. 12, conduits 102a, 102b can rotate around a common longitudinal axis 124. The fold mirrors 108 are facing first and second radial directions, often being opposed directions.

Optical conduits 102a and 102b may rotate continuously inside sleeve catheter 104. The imaging light runs through optical conduit 102a and provides an intra-vascular image of the artery, for instance by OCT. The ablating light runs through conduit 102b. The imaging and ablating lights can be used sequentially or simultaneously. The image is processed by a computer that identifies and localizes atherosclerotic plaques. Based on the information from the OCT imaging, the computer then determines when to fire the ablating light such that the light ablates specifically the plaque and does not damage the healthy area of the artery. This may be done by pulsing the ablating light on the plaque, when the optical path from the rotatable optical conduit 102b is oriented toward the plaque. The illustration of FIGS. 12A-12F show imaging light 112 and ablating light 114 being used simultaneously.

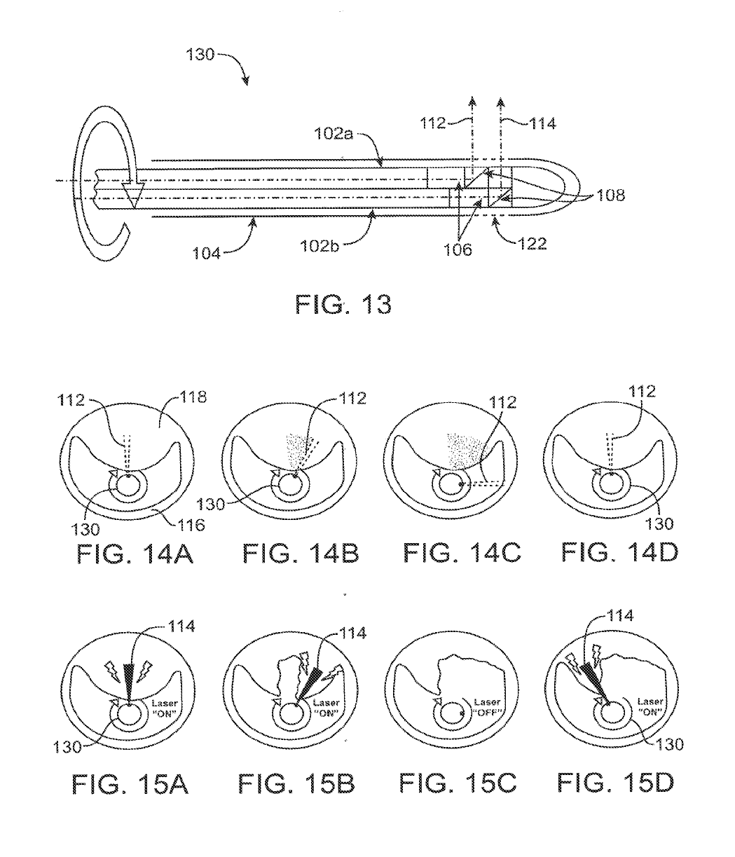

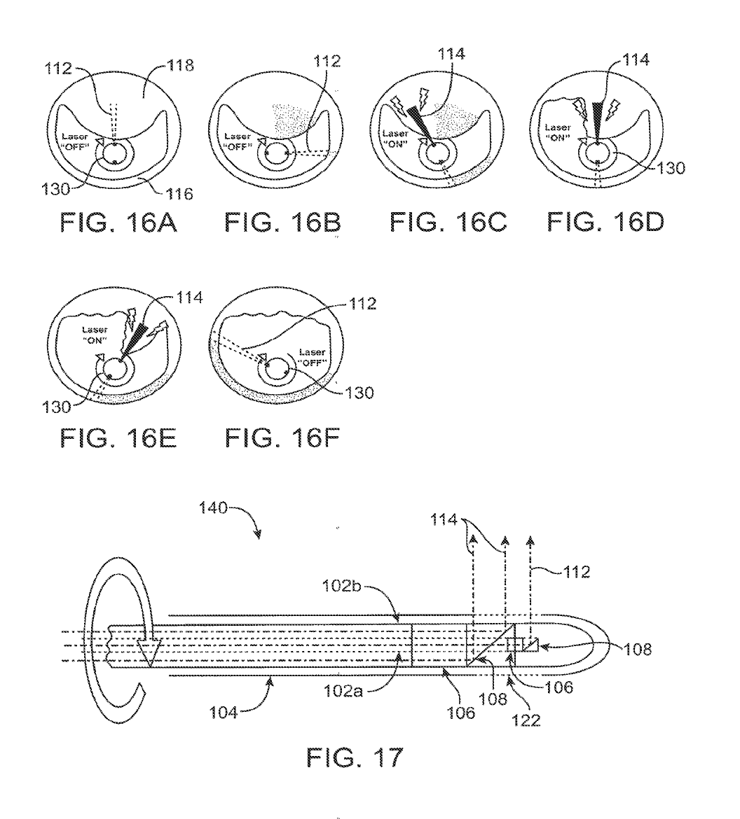

Preferred embodiment 130 may be understood with reference to FIGS. 13-16F. Two different optical conduits or bundles of optical conduits 102a and 102b, are used to convey the light energy for imaging, say imaging light 112, and the light energy for remodeling and/or ablating atherosclerotic plaques, say ablating light 114. The optical conduits 102a, 102b are housed inside a sleeve catheter or guidewire. Conduits 102a and 102b again may rotate around a common longitudinal axis, with their associated lenses 106 and fold mirrors 108 may be being axially staggered or separated and their optical paths facing either the same or different directions, depending on the configuration. The illustration of FIGS. 14A-14D and 15A-15D show the optical paths facing the same direction, and the imaging light 112 and the ablating light 114 being used sequentially. The illustration of FIG. 16 shows a different configuration: the imaging and optical paths face different, generally opposed directions, and imaging light 112 and ablating light 114 used simultaneously.

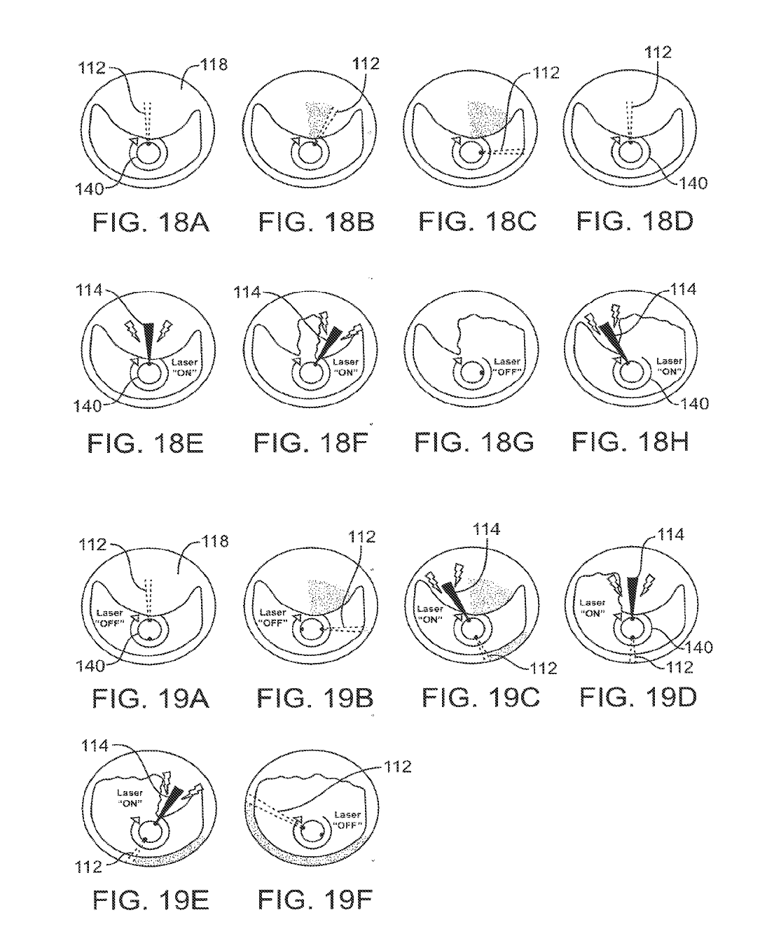

A preferred embodiment 140 is shown in FIGS. 17, 18A-18H, and 19A-19H. Two different optical probes or bundles of optical probes 102a and 102b, are used to convey light energy for imaging and light energy for remodeling and/or ablating atherosclerotic plaques. The optical probes are housed inside a sleeve catheter 104 or guidewire. Optical probes 102a, 102b rotate around a common longitudinal axis. Conduits 102a, 102b are coaxial and have staggered distal ends, and the associated fold mirrors 108 and optical paths may be facing either the same direction or different directions. The illustration of FIG. 17 shows the optical paths facing the same direction. The imaging and remodeling and/or ablating light energy can be used sequentially or simultaneously. The illustrations of FIGS. 18A-18H show the optical paths facing the same direction, and the imaging light and the ablating light being used sequentially. The illustrations of FIGS. 19A-19H show a different configuration: the optical paths face radially opposed directions, and imaging light 112 and ablating light 114 are used simultaneously.

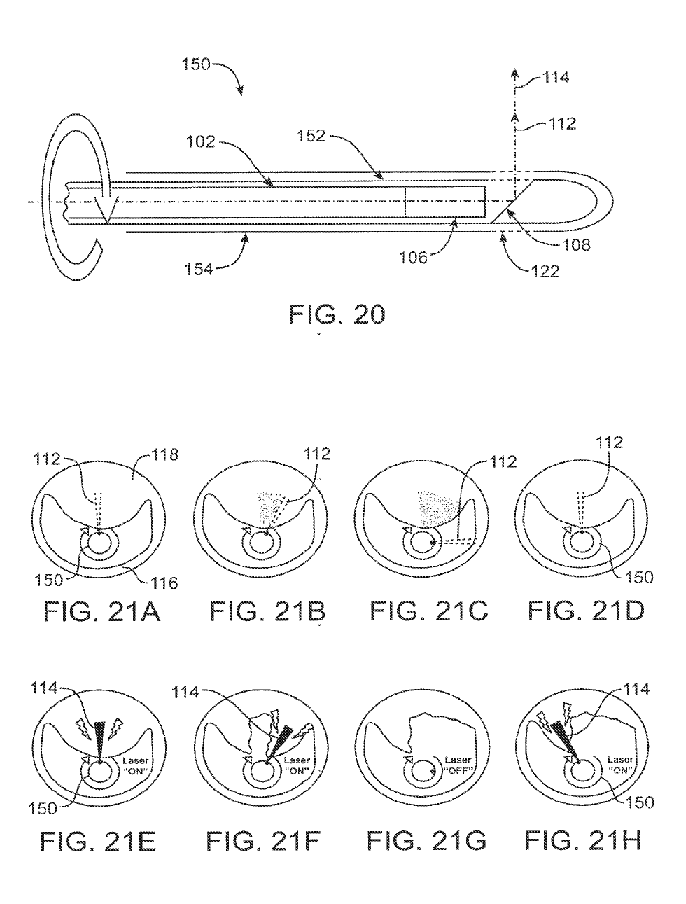

A preferred embodiment 150 is seen in FIGS. 20 and 21A-21H. In this embodiment, fold mirror 108 is independently movable relative to some or all of the other optical path elements (such as the optic conduit 102 and lens 106). In other words, fold mirror 108 is mounted on a rotatable sleeve 152 that can rotate around the rest of the optical conduits. Fold mirror 108 and sleeve 152 are attached to are rotatable relative to at least some of the remaining components of the optical path, and relative to a surrounding outer catheter sleeve 154. Sleeve 152 and mirror 108 rotate continuously inside outer sleeve catheter 154. Imaging light 112 runs through the optical conduits and provides an intra-vascular image of the artery, for instance by OCT. The image is processed by a computer that again identifies and localizes atherosclerotic plaques. Based on the information from the imaging system, the computer then determines when to fire the remodeling and/or ablating light 114 such that the light remodels and/or ablates specifically the plaque and does not damage the healthy area of the artery. The ablating light runs through the same optical conduits as the imaging light. The imaging and ablating lights may be used sequentially.

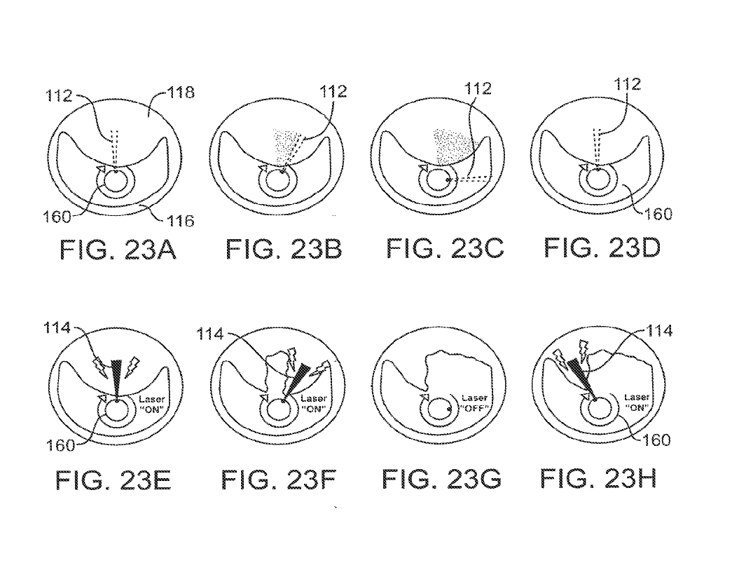

In preferred embodiment 160, as seen in FIGS. 22A-23H, the fold mirror 162 is again movable (typically rotatable) relative to other optical path components (optic fiber and lens). Fold mirror 162 is mounted on rotatable sleeve 152 that can rotate around the rest of the optical conduits. Two different optical conduits or bundles of optical conduits, 102a and 102b, are used to convey the light energy for imaging, imaging light 112, and the light energy for remodeling and/or ablating atherosclerotic plaques, ablating light 114. Conduits 102a and 102b are coaxial, with one optionally surrounding the other. The optical conduits 102a and 102b, and the sleeve mirror 162 are housed inside sleeve catheter 154 or guidewire. The conduits 102a, 102b, along with the associated optical paths and light energies 112, 114 may be reversed, so that imaging light may be disposed around the remodeling and/or ablating light energy. The sleeve mirror rotates continuously inside a sleeve catheter. The imaging light runs through 102a and provides with an intra-vascular image of the artery, for instance by OCT.

In order to ensure optimal imaging and ablation, as can be understood with reference to FIG. 22A, the portion or area of the mirror 166 that reflects the imaging light coming from 102a and the area or portion of mirror 164 that reflects ablating light 114 coming from 102b can have different properties, for example different reflectivity, focal shapes, or the like. The mirror reflecting the ablative energy can be convex and/or have a rough surface to disperse the ablative energy over an area broader than the area irradiated by the imaging light, in order to inhibit artery perforation and/or to ablate a larger area, while the mirror reflecting the imaging light can be flat and/or well polished to ensure precise and accurate imaging.

The remodeling and/or ablating light runs through conduit 102b. The imaging and ablating lights can be used sequentially or simultaneously. The illustration of FIGS. 23A-23H show imaging light 112 and ablating light 114 being used sequentially.

While the exemplary embodiments have been described in some detail, by way of example and for clarity of understanding, those of skill in the art will recognize that a variety of modification, adaptations, and changes may be employed. For example, a wide variety of mechanical, thermal, optical, ultrasonic or chemical working elements for treating atherosclerotic material, including those described in U.S. Pat. No. 6,120,516 (the full disclosure of which is incorporated herein by reference) might be employed in place of or in combination with the ablative laser energy described above. Aspects of the spectral diagnostic and treatment systems described in U.S. Pat. Nos. 5,304,173 and 6,117,128 (the full disclosures of which are incorporated herein by reference) may also be employed. Hence, the scope of the present invention should be limited solely by the appending claims.

What is claimed is:

1. An energy-treatment method, comprising:
(a) positioning a catheter-based device at a target position in a blood vessel, the catheter-based device in communication with an energy generator and a processor;
(b) using the catheter-based device, taking a plurality of temperature measurements at a plurality of points at the target position, the plurality of points substantially defining a helix;
(c) based on the plurality of temperature measurements, adjusting an output of the energy generator over a plurality of energization cycles; and
(d) energizing the catheter-based device over the plurality of energization cycles.

2. The method of claim 1, wherein the catheter-based device positioned in the blood vessel is in communication with a remodeling laser.

3. The method of claim 2, wherein the temperature measurements are taken by directing imaging light toward a first mirror disposed along a first optical path.

4. The method of claim 3, wherein the output of the energy generator is delivered along the first optical path.

5. The method of claim 3, wherein the output of the energy generator is delivered along a second optical path.

6. The method of claim 3, wherein the second optical path is offset from the first optical path.

7. The method of claim 6, wherein the first and second optical paths are axially offset.

8. The method of claim 6, wherein the first and second optical paths are circumferentially offset.

9. The method of claim 1, wherein the output of the energy generator is a remodeling energy that is less than an ablation energy.

10. A method of delivering energy comprising:
positioning a catheter-based energy delivery device with respect to a blood vessel;
using a first optical path, scanning along the blood vessel in a substantially helical pattern to obtain a plurality of temperature measurements;
processing the plurality of temperature measurements; and
based on the processed temperature measurements, adjusting an output of the catheter-based energy delivery device along the blood vessel.

11. The method of claim 10, further comprising energizing the catheter-based energy delivery device to direct a laser beam at a portion of the blood vessel.

12. The method of claim 11, wherein the laser beam is delivered along the first optical path.

13. The method of claim 11, wherein the laser beam is delivered along a second optical path.

14. The method of claim 13, wherein the second optical path is offset from the first optical path.

15. The method of claim 14, wherein the first and second optical paths are axially offset.

16. The method of claim 14, wherein the first and second optical paths are circumferentially offset.

17. The method of claim 10, wherein the output is a remodeling energy that is less than an ablation energy so that tissue associated with the blood vessel is not ablated.

18. The method of claim 10, wherein an optical coherence tomography system processes the plurality of temperature measurements.

19. An energy-treatment method, comprising:
(a) positioning a catheter-based device at a target position in a blood vessel, the catheter-based device in communication with an energy generator and a processor;
(b) using the catheter-based device, taking a plurality of temperature measurements at a plurality of points at the target position, the plurality of points substantially defining a helix;
(c) based on the plurality of temperature measurements, adjusting an output of the energy generator over a plurality of energization cycles to maintain a temperature of the target site of the blood vessel in a range of about 55 degrees Celsius to about 80 degrees Celsius; and
(d) energizing the catheter-based device over the plurality of energization cycles.

20. The energy-treatment method of claim 19, wherein the output is adjusted to maintain the temperature of the target sit in range of between about 50 degrees Celsius to about 60 degrees Celsius.
